(19) United States
(12) Patent Application Publication
(10) Pub. No.: US 2020/0402641 A1
(43) Pub. Date: Dec. 24, 2020

(54) SYSTEMS AND METHODS FOR CAPTURING AND PRESENTING LIFE MOMENT INFORMATION FOR SUBJECTS WITH COGNITIVE IMPAIRMENT

(71) Applicant: KONINKLIJKE PHILIPS N.V., EINDHOVEN (NL)

(72) Inventors: JORN OP DEN BUIJS, EINDHOVEN (NL); CHEVONE MARIE BARRETTO, LONDON (GB); PAUL MICHAEL FULTON, CAMBRIDGE (GB); ALAN WOOLLEY, CAMBRIDGE (GB); AART TIJMEN VAN HALTEREN, GELDROP (NL); NICOLAAS GREGORIUS PETRUS DEN TEULING, EINDHOVEN (NL); BENJAMIN EZARD, CAMBRIDGE (GB); ARUSHI ANEJA, YORK (GB)

(21) Appl. No.: 16/097,747

(22) PCT Filed: Apr. 14, 2017
(86) PCT No.: PCT/EP2017/059056

§ 371 Date: Oct. 30, 2018

Related U.S. Application Data
(60) Provisional application No. 62/331,506, filed on May 4, 2016.

(51) Int. Cl. G16H 20/70; G16H 40/67; G16H 10/60; G16H 50/20; G06Q 50/00; G06Q 10/10; A61B 5/16; A61B 5/00; A61B 5/11

(52) U.S. Cl. CPC G16H 20/70; G16H 40/67; G16H 10/60; G16H 50/20; G06F 3/0482; G06Q 10/1093; A61B 5/165; A61B 5/0077; A61B 5/1112; G06Q 50/01

(57) ABSTRACT

The present disclosure relates to a conversation facilitation system and method configured for capturing and presenting life moment information for a subject with cognitive impairment. The system and method comprises obtaining life moment information from one or more life moment capturing devices. The life moment information comprises information on daily activities experienced by the subject, including locations of daily activities and/or co-participants in the daily activities. The system and method comprises storing the life moment information for later review by the subject and/or a caregiver of the subject. The system and method comprises obtaining external data relating to the life moment information from one or more external data sources. The system and method comprises utilizing the life moment information and the external data to generate a life moment timeline. The life moment timeline is configured to display the life moment information to facilitate conversation between the subject and the caregiver.

### BACKGROUND

### 1. Field

The present disclosure relates to systems and methods for capturing and presenting life moment information for a subject with cognitive impairment.

### 2. Description of the Related Art

Reminiscence therapy (e.g., the review of past activities) is becoming a widely used psychosocial intervention in subjects with cognitive impairment and dementia. Currently, reminiscence therapy is facilitated by manually keeping journals or diaries.

### SUMMARY

Accordingly, it is an aspect of one or more embodiments of the present disclosure to provide a conversation facilitation system configured to capture and present life moment information as reminiscence therapy for a subject with cognitive impairment. The system comprises one or more hardware processors and/or other components. The one or more hardware processors are configured by machine-readable instructions to obtain life moment information from one or more life moment capturing devices. The life moment information comprises information on daily activities experienced by the subject, including locations of the daily activities and/or co-participants in the daily activities. The life moment information further comprises physiological information of the subject derived from biometric signals, including physical activity, mood, and/or emotion. The one or more hardware processors are further configured to store the life moment information to electronic storage for later review by the subject and/or a caregiver of the subject. The one or more hardware processors are further configured to obtain external data relating to the life moment information from one or more external data sources. The external data sources comprise one or more of public records, social media data sources, web-based data sources, or current moment data sources. The one or more hardware processors are further configured to utilize the life moment information and the external data to generate a life moment timeline. The life moment timeline is configured to display the life moment information.

It is yet another aspect of one or more embodiments of the present disclosure to provide a method for capturing and presenting life moment information as reminiscence therapy to a subject with cognitive impairment. The method comprises one or more hardware processors configured to execute machine-readable instructions and/or other components. The method comprises obtaining life moment information from one or more life moment capturing devices. The life moment information comprises information on daily activities experienced by the subject, including locations of the daily activities and/or co-participants in the daily activities. The life moment information further comprises physiological information of the subject derived from biometric signals, including physical activity, mood, and/or emotion. The method further comprises storing the life moment information to electronic storage for later review by the subject and/or a caregiver of the subject. The method further comprises obtaining external data relating to the life moment information from one or more external data sources. The external data sources comprise one or more of public records, social media data sources, web-based data sources, or current moment data sources. The method further comprises utilizing the life moment information and the external data to generate a life moment timeline. The life moment timeline is configured to display the life moment information.

It is yet another aspect of one or more embodiments of the present disclosure to provide a conversation facilitation system configured to capture and present life moment information as reminiscence therapy for a subject with cognitive impairment. The system comprises means for obtaining life moment information from one or more life moment capturing devices. The life moment information comprises information on daily activities experienced by the subject, including locations of the daily activities and/or co-participants in the daily activities. The life moment information further comprises physiological information of the subject derived from biometric signals, including physical activity, mood, and/or emotion. The system further comprises means for storing the life moment information to electronic storage for later review by the subject and/or a caregiver of the subject. The system further comprises means for obtaining external data relating to the life moment information from one or more external data sources. The external data sources comprise one or more of public records, social media data sources, web-based data sources, or current moment data sources. The system further comprises means for utilizing the life moment information and the external data to generate a life moment timeline. The life moment timeline is configured to display the life moment information.

These and other aspects, features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. In some embodiments, the structural components illustrated herein are drawn in proportion. It is to be expressly understood, however, that the drawings are for the purpose of illustration and description only and are not a limitation of the present disclosure. In addition, it should be appreciated that structural features shown or described in any one embodiment herein can be used in other embodiments as well. It is to be expressly understood, however, that the drawings are for the purpose of illustration and description only and are not intended as a definition of the limits of the present disclosure.

### BRIEF DESCRIPTION OF THE DRAWINGS

FIG. 1 illustrates a conversation facilitation system configured to capture and present life moment information for a subject with cognitive impairment, in accordance with one or more embodiments;

FIG. 2 illustrates an exemplary sensor configuration, in accordance with one or more embodiments;

FIG. 3 illustrates an exemplary life moment timeline configuration, in accordance with one or more embodiments;

FIG. 4 illustrates an exemplary method for capturing and presenting key events from life moment information, in accordance with one or more embodiments; and

FIG. 5 illustrates a method for capturing and presenting life moment information to a subject with cognitive impairment, in accordance with one or more embodiments.

### DETAILED DESCRIPTION OF THE EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, e.g., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (e.g., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

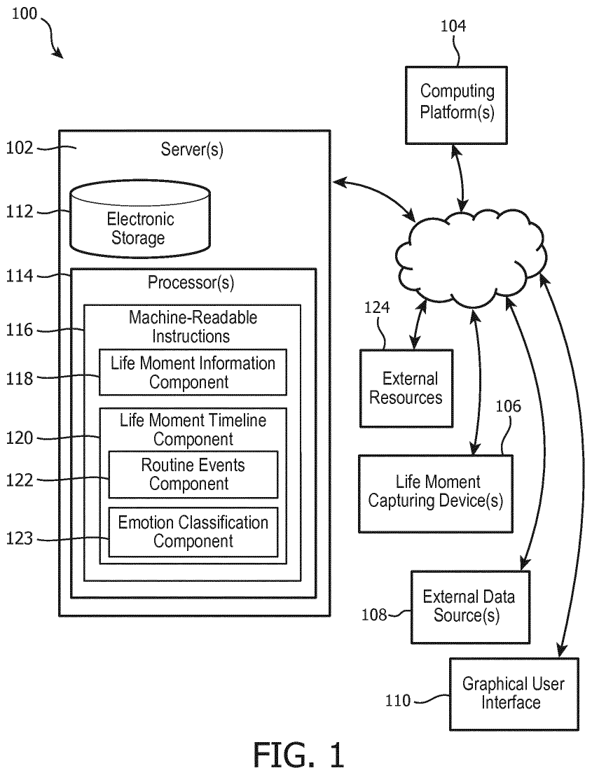

FIG. 1 illustrates a conversation facilitation system 100 configured to capture and present life moment information for a subject with cognitive impairment, in accordance with one or more embodiments. In some embodiments, life moment information may include information related to daily activities experienced by the subject, including locations of the daily activities, co-participants in the daily activities, and/or other information.

About 10-20% of older adults have mild cognitive decline, in many cases this may be a precursor of Alzheimer's disease and other forms of dementia. Loss of short term memory is a problem for people with dementia and other cognitive impairment. Memory of places, names, and faces may all be affected, as can memory for recent and/or upcoming events. A consequence of such memory loss may include disorientation, where a subject may become anxious and/or confused because they are not sure of the time, where they are, and/or what they should be doing. Caregivers for people in this situation and/or similar situations often have to provide necessary reassurance, usually through the use of visual and/or verbal cues to guide memory recall and/or conversation.

Reminiscence therapy (e.g., the review of past activities) is a widely used psychosocial intervention that emphasizes recalling and/or re-experiencing one's own life events as a way of improving engagement, attitude, and/or general wellbeing of subjects with cognitive impairments and/or other memory problems. Cognitive impairments may include memory conditions, neurodegenerative diseases such as dementia or Alzheimer's disease, and/or other cognitive impairments. Conventional approaches to reminiscence therapy include keeping hand written journals, diaries, photo albums, and/or playing familiar songs, however these approaches are reliant on such keepsakes being readily accessible, if recorded at all, and/or manually having to input data around key life moments in the day of the subject. These approaches may be able to stimulate personal discussion, but only for documented events and therefore are not tailored for recording, storing, and/or reminiscing of daily activities in the subject's life.

Exemplary embodiments of the present system address problems that specifically arise out of subjects forgetting to enter the information. Further, exemplary embodiments of the present system may alleviate problems with prior art electronic systems in situations where automatically capturing a stream of photographs generates redundant data, which is cumbersome for the user to have to manually sort through at a later moment in time. In addition, merely collecting and presenting data around certain moments in the day does not tell a story. Exemplary embodiments of the present system engage a subject interactively, (e.g., presents questions to the subject and/or caregiver for review such as “Who did you speak to today?”). Further, exemplary embodiments of the present system may stimulate the subject through immersive media, creating a sense of immersion (e.g., captured multimedia reflects the mood of the subject). Accordingly, it is an aspect of one or more embodiments of the present disclosure to provide an electronic reminiscence diary automatically generated without creating a large amount of redundant data that is not helpful to the user.

Exemplary embodiments of the present disclosure overcome the problems of current approaches to reminiscence therapy by capturing life moment information for a subject with cognitive impairment then identifying key events from the subject's life moment information based on sensor data analysis, physiological measurements, and/or other information. Key events are life events or moments in a subject's day that are noteworthy or otherwise important for the subject to remember. By way of non-limiting example, key events may include having lunch with a friend, visiting with a family member, watching a favorite television show, talking on the phone, shopping, attending church, seeing a medical practitioner, engaging in routine daily activities such as bathing, dressing, eating, taking daily medication, and/or other noteworthy life moments. Recording and/or storage of multimedia content may be facilitated by one or more life moment capturing devices. Life moment capturing devices may be configured to compile data into a timestamped record. One or more sensors may be configured to detect a change in activity by the subject and/or detect whether the subject is engaging in a key event. Such detection may prompt the life moment capturing device(s) to capture, record, collect, store, and/or facilitate other processes, in accordance with one or more embodiments.

Exemplary embodiments of the present disclosure are beneficial for a caregiver of a subject with cognitive impairment, as it allows the caregiver to spend time with the subject more efficiently, decreasing the workload for the caregiver. Key event data can be shared with one or both of professional and/or familial caregivers, thereby improving communication efficiency. A caregiver may include caregivers such as a friend, family member, neighbor, lifestyle coach, fitness coach, and/or another person involved in the subject's life or daily activities. As another example, a caregiver may include a health professional such as a nurse, doctor, general practitioner, care provider, mental health professional, heath care practitioner, physician, dentist, pharmacist, physician assistant, advanced practice registered nurse, surgeon, surgeon's assistant, athletic trainer, surgical technologist, midwife, dietitian, therapist, psychologist, chiropractor, clinical officer, social worker, phlebotomist, occupational therapist, physical therapist, radiographer, radiotherapist, respiratory therapist, audiologist, speech pathologist, optometrist, operating department practitioner, emergency medical technician, paramedic, medical laboratory scientist, medical prosthetic technician, and/or other human resources trained to provide some type of health care service.

Exemplary embodiments of the present disclosure include a system configured to facilitate automatic capturing and presentation of life moment information for a subject with cognitive impairment. The system is configured to record and/or identify key events, life moments, locations, and/or people associated with a subject, and present this information to the subject in the form of a timeline and/or other visual representation. The timeline may be presented via a graphical user interface (GUI) on a personal computing device, including a mobile phone, smartphone, laptop computer, desktop computer, tablet computer, PDA (e.g., personal digital assistant, personal data assistant, and/or other mobile electronic device), a netbook, a handheld PC, a smart TV, and/or other personal computing devices. The timeline may facilitate conversation between the subject and a caregiver by reviewing key events and people met, which may result in a better recollection of those events and/or people. This method of reminiscence therapy may increase confidence and/or reduce anxiety related to orientation and situational awareness, with the potential to improve short-term memory and/or delay the progression of cognitive decline, among other possible advantages. By presenting a subject with familiar information and/or activity reminders, the present disclosure may prevent distressing and/or anxious situations which otherwise may result in use of emergency services. The system(s) and/or method(s) as described herein may benefit a subject's prospective memory, such that reviewing the timeline of prior events may improve the subject's recollection of similar upcoming events.

As shown in FIG. 1, in some embodiments, system 100 may include one or more servers 102, one or more computing platforms 104, one or more life moment capturing devices 106, one or more external data sources 108, a graphical user interface (GUI) 110, and/or other components. In some embodiments, server(s) 102, computing platform(s) 104, life moment capturing device(s) 106, external resources 124, external data source(s) 108, and/or graphical user interface 110 may be operatively linked via one or more electronic communication links. For example, such electronic communication links may be established, at least in part, via a network such as the Internet and/or other networks. It will be appreciated that this is not intended to be limiting, and that the scope of this disclosure includes embodiments in which server(s) 102, computing platform(s) 104, life moment capturing device(s) 106, external resources 124, external data source(s) 108, and/or graphical user interface 110 may be operatively linked via some other communication media.

Server(s) 102 include electronic storage 112, one or more processors 114, and/or other components. Server(s) 102 may include communication lines, or ports to enable the exchange of information with a network and/or other computing platforms. Illustration of server(s) 102 in FIG. 1 is not intended to be limiting. Server(s) 102 may include a plurality of hardware, software, and/or firmware components operating together to provide the functionality attributed herein to server(s) 102. For example, server(s) 102 may be implemented by a cloud of computing platforms operating together as server(s) 102.

Server(s) 102 is configured to communicate with computing platform(s) 104 according to a client/server architecture, a peer-to-peer architecture, and/or other architectures. Computing platform(s) 104 may include one or more processor(s) 114 configured to execute machine-readable instructions 116. Machine-readable instructions 116 may be configured to enable an expert or user associated with computing platform(s) 104 to interface with system 100, life moment capturing device(s) 106, external resources 124, graphical user interface 110, and/or external data source(s) 108, and/or provide other functionality attributed herein to computing platform(s) 104. By way of non-limiting example, computing platform(s) 104 may include one or more of a desktop computer, a laptop computer, a handheld computer, a tablet computing platform, a NetBook, a Smartphone, a gaming console, and/or other computing platforms.

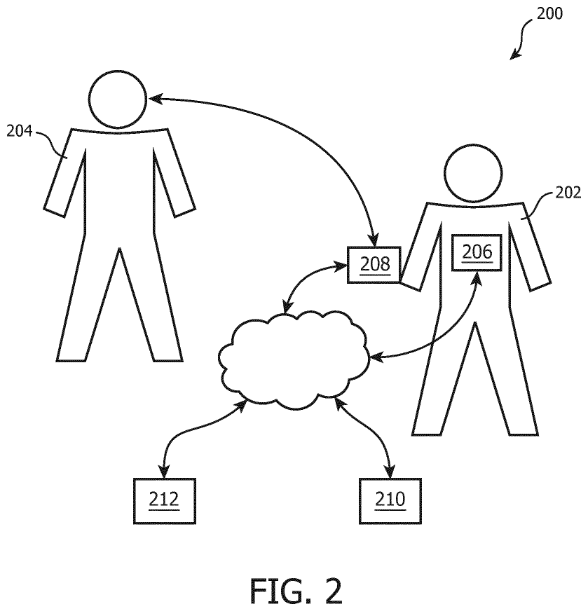

As described above, in some embodiments, system 100 comprises one or more life moment capturing device(s) 106. One embodiment of the present system relates to a wearable system configured to automatically capture information related to “life moments” during a subject's daily activity. Life moment information may be captured and/or identified using a variety of sensor data and multimedia information (e.g., images, video, sound, and/or other multimedia). Life moment capturing device(s) 106 may include one or more of wearable devices, biometric devices, multimedia devices, location devices, and/or other life moment capturing devices and/or sensors. Sensor data generated by such devices may include information gathered from wearable sensors such as blood pressure sensors, heart rate sensors, skin conductance sensors, a Philips Health watch, an Apple watch, a Philips GoSafe, a Global Positioning System (GPS), physiological sensors, weight sensors, and/or other sensors and/or devices. As used herein, the term “physiological” encompasses “psychological” and/or “emotional”, however, for purposes of brevity, “psychological” and “emotional” may not always be stated. Physiological data includes one or more of heart rate information, electrodermal activity, blood pressure information, body temperature information, pulse rate information, respiration rate information, heart rate variability information, skin temperature information, skin conductance response information, activity data information, movement information, pedometer data, accelerometer data, electroencephalogram (EEG) data (e.g., brain activity), electromyogram (EMG) data (e.g., muscle activity), electrocardiogram (ECG or EKG) data (e.g., heart activity), stamina information, and/or other physiological information. Physiological information associated with an emotional state of the subject may be monitored using life moment capturing device(s) 106 such as vital signs sensors (e.g., Philips Vital Signs Camera, heart rate monitor, galvanic skin response monitor, and/or other vital signs equipment), sound and video recorders (e.g. voice recorder), cameras, wearable cameras, wearable sensors (e.g., a smartwatch, Philips Lifeline pendant, and/or other sensors), in-body sensors (e.g., electronic pills that are swallowed), and/or other physiological state monitors. An exemplary life moment capturing device sensor configuration is illustrated FIG. 2.

FIG. 2 illustrates an exemplary sensor configuration 200, in accordance with one or more embodiments. In some embodiments, a subject 202 may have emotional state information, physiological state information, location information, and/or other subject information monitored by sensors configured to provide output signals that convey information related to the emotional state of subject 202, physiological state of subject 202, location of subject 202, and/or other information associated with subject 202. In some embodiments, one or more co-participants 204 may have emotional state information obtained by sensors configured to provide output signals that convey information related to the emotional state information of co-participant(s) 204 and/or other information about co-participant(s) 204. In some embodiments, co-participant(s) 204 may have emotional state information obtained by sensors configured to monitor subject 202. By way of non-limiting example, a sensor worn by subject 202 may record the voice(s) of co-participant(s) 204 and facilitate identification of co-participant(s) 204. Sensor configuration 200 may include one or more sensors, devices, and/or other components that are the same as or similar to life moment capturing device(s) 106 (as described in connection with FIG. 1), in accordance with one or more embodiments.

Sensor configuration 200 may include one or more of subject wearable sensors 206, subject biometric sensors 208, subject multimedia sensors 210, subject location sensors 212, and/or other sensors, in accordance with one or more embodiments. Subject wearable sensors 206 may include one or more of a smartwatch, Philips Lifeline pendant, Philips Health watch, Philips GoSafe, and/or other wearable sensors. Subject biometric sensors 208 may include one or more of heart rate sensors, galvanic skin response sensors, skin conductance sensors, accelerometer, blood pressure sensors, EKG/cardiac monitors, pedometers, and/or other biometric sensors. Subject multimedia sensors 210 may include one or more of a camera, a video recorder, a wearable camera, a personal computing device equipped with an optical instrument for recording images, Philips Vital Signs Camera, audio and voice recorders, and/or other multimedia equipment. Subject location sensors 212 may include one or more of a Global Positioning System (GPS) device, a personal computing device equipped with a location tracker, a navigation system, a personal navigation assistant (PNA), and/or other location sensors.

Returning to FIG. 1, in some embodiments, system 100 comprises one or more external data source(s) 108. In current approaches to display reminiscence therapy features, automatically collected context and activity data is not always user friendly (e.g., one piece of data is not obviously connected to a subsequent piece of data and neither piece data is presented in a visually pleasing way). System 100 provides an intelligent combination of that data with associated selected data from external data source(s) 108 to make the presented output more personal, social, and/or more user friendly and aesthetically pleasing. By way of non-limiting example, GPS data associated with a subject's location may be integrated with images and/or data from public databases (e.g., Google Street View images) to provide a more attractive user interface and or more information than the captured data alone. In another non-limiting example, key event(s) associated with a subject's life moments may be matched to news stories, personal social media feeds, public photos, and/or other data from external data source(s) relating to the key event(s) experienced by the subject. The external data may be integrated in the display of the subject's key event(s) information for a more comprehensive reviewing experience. External data source(s) 108 may include one or more of internet, websites, public records, social media data sources, web-based data sources, news data sources, weather data sources, Google Street View, and/or other external data sources.

In some embodiments, external data sources may include external resources 124. In some embodiments, external resources 124 are standalone components of system 100. External resources 124 include sources of information, hosts, and/or providers of electronic health records (EHRs), external entities participating with system 100, external data analysis resources, and/or other resources. For example, in system 100, external resources 124 are configured to aggregate and/or post-process multimedia content (e.g., content generated by life moment capturing device(s) 106 and/or other external data source(s) 108) into a visual representation of the data presented via graphical user interface 110 (described below). Post-processing of multimedia content may include aggregating content from external data source(s) 108 such as web search, mapping services, social networking sites, personal calendar and e-mail, and/or other data sources. By way of non-limiting example, the visual representation may include a timeline, a slideshow of pictures with background music, merged video footage, news and weather information, and/or other life moment information.

As described above, in some embodiments, system 100 comprises graphical user interface (GUI) 110. Graphical user interface 110 is displayed via computing platform(s) 104 and/or other devices. Graphical user interface 110 is configured to display the information from life moment capturing device(s) 106, external data source(s) 108, and/or other information to the subject, the caregiver, and/or other users. Graphical user interface 110 may include an application programming interface (API), in accordance with one or more embodiments. Graphical user interface 110 may be configured to generate and maintain a user interface that can be incorporated with a site (e.g., a web site and/or a mobile site) and/or app provided by server(s) 102 and/or other servers. The user interface may serve as a graphical interface for a user visiting the site or utilizing the app. The graphical user interface (GUI) may be displayed via a personal computing device, including a mobile phone, smartphone, laptop computer, desktop computer, tablet computer, PDA (e.g., personal digital assistant, personal data assistant, and/or other mobile electronic device), a netbook, a handheld PC, and/or other personal computing devices.

In some embodiments, graphical user interface 110 is configured to provide an interface between system 100 and user(s) (e.g., subjects, caregivers, and/or other users) through which user(s) may provide information to and receive information from system 100. This enables data, results, and/or instructions and any other communicable items, collectively referred to as “information,” to be communicated between the user(s) and one or more of processor(s) 114, and/or electronic storage 112. Examples of interface devices suitable for inclusion in graphical user interface 110 include a keypad, buttons, switches, a keyboard, knobs, levers, a display screen, a touch screen, speakers, a microphone, an indicator light, an audible alarm, a printer, and/or other devices. It is to be understood that other communication techniques, either hard-wired or wireless, are also contemplated by the present disclosure as graphical user interface 110. For example, the present disclosure contemplates that graphical user interface 110 may be integrated with a removable storage interface provided by electronic storage 112. In this example, information may be loaded into system 100 from removable storage (e.g., a smart card, a flash drive, a removable disk, etc.) that enables the user(s) to customize the implementation of system 100. Other exemplary input devices and techniques adapted for use with system 100 as graphical user interface 110 include, but are not limited to, an RS-232 port, RF link, an IR link, modem (telephone, cable or other). In short, any technique for communicating information with system 100 is contemplated by the present disclosure as graphical user interface 110.

Electronic storage 112 may comprise non-transitory storage media that electronically stores information. The electronic storage media of electronic storage 112 may include one or both of system storage that is provided integrally (e.g., substantially non-removable) with server(s) 102 and/or removable storage that is removably connectable to server(s) 102 via, for example, a port (e.g., a USB port, a firewire port, and/or other types of ports) or a drive (e.g., a disk drive and/or other types of drive). Electronic storage 112 may include one or more of optically readable storage media (e.g., optical disks and/or other optically readable storage media), magnetically readable storage media (e.g., magnetic tape, magnetic hard drive, floppy drive, and/or other magnetically readable storage media), electrical charge-based storage media (e.g., EEPROM, RAM, and/or other electrical charge-based storage media), solid-state storage media (e.g., flash drive, and/or other solid-state storage media), and/or other electronically readable storage media. Electronic storage 112 may include one or more virtual storage resources (e.g., cloud storage, a virtual private network, and/or other virtual storage resources). Electronic storage 112 may store software algorithms, information determined by processor(s) 114, information received from server(s) 102, information received from computing platform(s) 104, information received from life moment capturing device(s) 106, information received from external data source(s) 108, information received from graphical user interface 110, information received from external resources 124, information associated with machine-readable instructions 116, information associated with life moment information component 118, information associated with life moment timeline component 120, information associated with routine events component 122, information associated with emotion classification component 123, and/or other information that enables system 100 to function as described herein.

As described above, server(s) 102 include one or more processor(s) 114. Processor(s) 114 are configured to execute machine-readable instructions 116. Machine-readable instructions 116 may include one or more of life moment information component 118, life moment timeline component 120, routine events component 122, emotion classification component 123, and/or other machine-readable instruction components. Processor(s) 114 may be configured to provide information processing capabilities in server(s) 102 and/or in system 100 as a whole. As such, processor(s) 114 may include one or more of a digital processor, an analog processor, a digital circuit designed to process information, an analog circuit designed to process information, a state machine, and/or other mechanisms for electronically processing information. Although processor(s) 114 is shown in FIG. 1 as a single entity, this is for illustrative purposes only. In some embodiments, processor(s) 114 may include a plurality of processing units. These processing units may be physically located within the same device, or processor(s) 114 may represent processing functionality of a plurality of devices operating in coordination. The processor(s) 114 may be configured to execute machine-readable instruction components 118, 120, 122, 123, and/or other machine-readable instruction components. Processor(s) 114 may be configured to execute machine-readable instruction components 118, 120, 122, 123, and/or other machine-readable instruction components by software; hardware; firmware; some combination of software, hardware, and/or firmware; and/or other mechanisms for configuring processing capabilities on processor(s) 114. As used herein, the term “machine-readable instruction component” may refer to any component or set of components that perform the functionality attributed to the machine-readable instruction component. This may include one or more physical processors during execution of processor readable instructions, the processor readable instructions, circuitry, hardware, storage media, or any other components.

It should be appreciated that although machine-readable instruction components 118, 120, 122, and 123 are illustrated in FIG. 1 as being implemented within a single processing unit, in embodiments in which processor(s) 114 includes multiple processing units, one or more of machine-readable instruction components 118, 120, 122, and/or 123 may be implemented remotely from the other machine-readable instruction components. The description of the functionality provided by the different machine-readable instruction components 118, 120, 122, and/or 123 described below is for illustrative purposes, and is not intended to be limiting, as any of machine-readable instruction components 118, 120, 122, and/or 123 may provide more or less functionality than is described. For example, one or more of machine-readable instruction components 118, 120, 122, and/or 123 may be eliminated, and some or all of its functionality may be provided by other ones of machine-readable instruction components 118, 120, 122, and/or 123. As another example, processor(s) 114 may be configured to execute one or more additional machine-readable instruction components that may perform some or all of the functionality attributed below to one of machine-readable instruction components 118, 120, 122, and/or 123.

In some embodiments, life moment information component 118 is configured to obtain life moment information from life moment capturing device(s) 106. Life moment information component 118 is configured to store the life moment information (e.g., electronic storage 112) for later review by the subject and/or a caregiver of the subject. In some embodiments, life moment information component 118 is configured to obtain external data relating to the life moment information from external data source(s) 108. In some embodiments, life moment information component 118 may be configured to continuously and/or near continuously obtain multimedia content from life moment capturing device(s) 106 (such as a wearable camera) and/or from external data sources 108. In some embodiments, life moment information component 118 obtains and stores multimedia content in response to identification and/or detection of key event(s). As described above, key events are life events or moments in a subject's day that are noteworthy or otherwise important for the subject to remember.

In some embodiments, key event(s) are detected and/or identified by life moment information component 118 based on multiple input signals from life moment capturing devices 106 and/or other devices (e.g., audio to identify an interaction with another person, galvanic skin response (GSR) to identify changes in emotion, GPS to identify when a stationary period follows movement, accelerometer data to detect physical activity, and/or other input signals). By way of non-limiting example, GPS technology may continuously capture a subject's location data. Life moment information component 118 may be configured to identify life moment information as a key event responsive to the location data indicating a change in the subject's location and/or other indications. In another non-limiting example, accelerometers may be worn by the subject to monitor physical activity. Key event activities may be detected by the subject's intensity and/or duration of acceleration exceeding a threshold value of intensity and/or duration. In another non-limiting example, detection of key event(s) may be based on biomedical and/or biometric signals such as detection of spikes or measured increases in a subject's galvanic skin response (GSR), heart rate, and/or other biometric data. Life moment capturing device(s) 106 may be configured to record multimedia for a user-defined duration, in specified intervals, in response to subject activity, and/or in accordance with other configurations of duration. In another non-limiting example, buffered multimedia content (e.g., content recorded before a key event) may be stored responsive to key event(s) being identified.

Life moment timeline component 120 is configured to utilize the life moment information and the external data to generate a visual presentation of key event(s) information. The life moment information displayed by the life moment timeline may facilitate conversation between the subject and the caregiver. Key event information may include time and date information associated with a key event, location information associated with the key event, co-participants associated with the key event, activities associated with the key event, multimedia associated with the key event, physiological information of the subject associated with the key event, and/or other information associated with the key event.

Time and date information may include the date associated with a key event, the time of day the key event occurred, the duration of the key event, a schedule of activities and/or key events, and/or other information. Routine events component 122 may be configured to detect a subject's routine activities based on past key event(s) and store those routine activities to the timeline. In some embodiments, a predictive feature of the routine events may be configured to suggest activities and/or provide a reminder to the subject. By way of non-limiting example, responsive to the subject going to a dance class every Wednesday evening, the event is detected as a routine event and a reminder notification may be sent to the subject prior to the next Wednesday evening dance class event. Such notifications and/or other features of routine events component 122 may aid the subject with prospective memory for upcoming events, as reminiscence of past activities may remind the subject of upcoming activities.

Location information may include geographical location, structures associated with the location, whether the location was a residence, business, and/or other type of location, and/or other location information associated with a key event. Life moment timeline component 120 may be configured to describe journeys, meetings, tasks, and/or other activities that the subject was engaged in that day. In some embodiments, key event(s) may occur within the home environment. Life moment timeline component 120 may be configured to identify key facts and present them to the subject and/or caregiver for review via the graphical user interface (e.g., “Who did you speak to today?”, “What have you watched on TV?”, “Which parts of your daily routine have you completed?”, and/or other key facts).

Co-participant information may include people the subject interacted with, profile information (e.g., name, relationship to the subject, and/or other information) about those people, and/or other co-participant information associated with the key event. In some embodiments, life moment timeline component 120 may be configured for face recognition of recorded images to aid subjects in name recollection. By way of non-limiting example, a family member and/or other caregiver may label images via the GUI. Image labeling may facilitate calibration of an algorithm for future face and/or image recognition, in accordance with one or more embodiments.

Multimedia associated with the key event may include photos, videos, images, audio, web content, and/or other multimedia content. In some embodiments, life moment information may be matched to web-sourced data to enhance presentation to the user. Multimedia content may be filtered using image classification software, in accordance with one or more embodiments. By way of non-limiting example, if a relevant image (such as a meal) is detected by the classifier, the image may be labeled and/or included as a life moment, key event, and/or as other information. In some embodiments, life moment timeline component 120 may be configured to emphasize changes of context (e.g., location, social situation, mood, weather, lighting, personal motivations that cross multiple events such as “planning to go out”, and/or other changes of context). In some embodiments, key events may be categorized into different and/or same types (e.g., a walk in the park and/or a walk by the river may both be categorized as a walk in the local area).

Physiological information associated with the key event may include classification of the subject's physiological and/or emotional state based on the recorded physiological data associated with the subject's life moments. Emotion classification component 123 may be configured to classify the physiological and/or emotional state of the subject. Emotion classification component 123 may be configured to process and/or post-process multimedia content based on the classification of the subject's physiological and/or emotional state. For example, facial expressions may be assessed by the system to determine mood, emotion, pain, tiredness, and/or other physiological state and/or emotional state information.

In some embodiments, emotion classification component 123 may be configured to post-process multimedia content associated with key event(s). Post-processing of multimedia content may be based on a classification of the subject's mood (e.g., photographs recorded during positive, happy moments may be post-processed such that bright colors are amplified, photographs recorded during negative, sad moments may be post-processed to be displayed in black and white, videos recorded during negative, sad moments may be post-processed such that sad music is applied, and/or other multimedia post-processing according to emotional state). In some embodiments, post-processing may be based on the emotion of persons the subject interacted with. Image and/or speech processing may be used to classify the emotion of the person encountered by the subject.

Responsive to identifying key event(s) from the life moment information of a subject, the emotion of the subject may be classified (e.g., as negative, neutral, or positive) based on biometric information of the subject during the key event. Various types of wearable and/or passive sensors (e.g., activity sensors, posture sensors, GSR sensors, heartrate sensors, blood pressure sensors, image sensors, speech sensors, and/or other passive sensors) may be configured to provide output signals conveying information to facilitate categorizing emotions such as angry, sad, happy, and/or other emotions.

In some embodiments, the multimedia captured during key event(s) may be post-processed according to the categorized emotional state of the subject. In some embodiments, emotion classification may be facilitated by an external classification algorithm. In some embodiments, post-processing instructions may be provided by a database. The emotion classification algorithm may be an “out-of-the-box” algorithm, or an algorithm that requires calibration by a user. By way of non-limiting example, emotion classification algorithms as described in “Real-Time Stress Detection by Means of Physiological Signals”, by Alberto de Santos Sierra, et al. (2011, DOI: 10.5772/18246) may be used.

Responses in physiological parameters after stimuli may be highly individual. By way of non-limiting example, a first step of emotion classification may include extracting a physiological exemplar and/or template from the subject (e.g., the subject is exposed to stress stimuli such as hyperventilation and/or talk preparation to induce different emotions and/or stress levels). Parameters may be extracted from physiological measurements associated with the subject (e.g., heart rate, galvanic skin response, and/or other measurements associated with a subject). Parameters may be extracted from the subject's measurements at baseline and/or in the period after inducing the stimuli. Parameters may include mean and/or standard deviation of the subject's heart rate, galvanic skin response, and/or other physiological parameters. In some embodiments, a database of individual response characteristics, features, and/or parameters to physiological stimuli may be generated. By way of non-limiting example, a second step of emotion classification may include determining stress level(s) and/or emotional state(s) associated with the subject using recognized classification methods (e.g., k-Nearest Neighbors algorithm (k-NN), linear discriminant analysis (LDA), support vector machines (SVMs), support vector networks, and/or other methods).

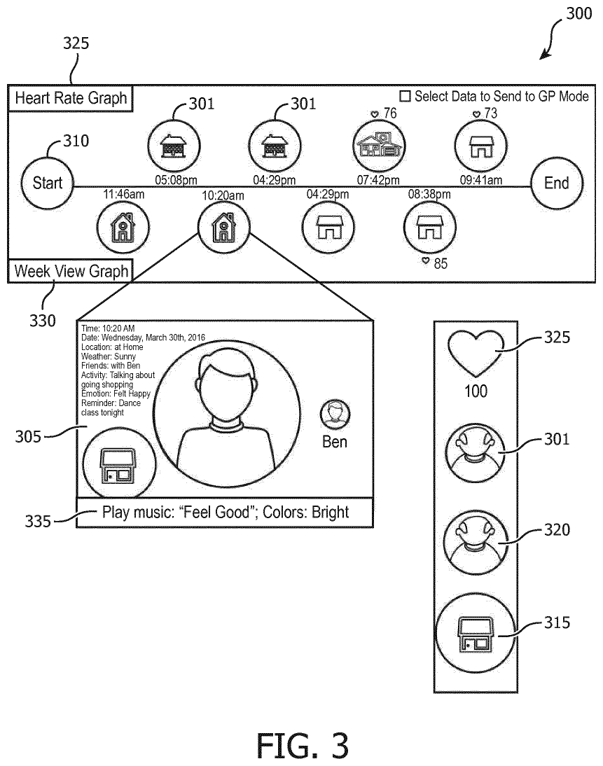

FIG. 3 illustrates an exemplary life moment timeline configuration 300, in accordance with one or more embodiments. Life moment timeline 300 may be configured to present life moment information as reminiscence therapy to a subject with cognitive impairment, in accordance with one or more embodiments. Life moment timeline 300 may be displayed via a graphical user interface. Life moment timeline 300 may be configured to utilize the life moment information and the external data to generate a life moment timeline. The life moment timeline may be configured to display the life moment information as reminiscence therapy to facilitate conversation between the subject and the caregiver. Life moment timeline 300 may include one or more fields, modules, and/or other components that are the same as or similar to life moment timeline component 120 (as described in connection with FIG. 1), in accordance with one or more embodiments.

In some embodiments, life moment timeline 300 may include one or more interactive fields. The interactive fields may include one or more of an event field 305, a period field 310, a location field 315, a friend field 320, a health field 325, a routine field 330, an emotion field 335, and/or other fields. Life moment timeline 300 may be configured to display key event information associated with one or more key events identified from life moment information the same as or similar to life moment information component 118 (as described in connection with FIG. 1), in accordance with one or more embodiments.

In some embodiments, life moment timeline 300 may include one or more thumbnail(s) 301. Thumbnail(s) 301 may include reduced-size versions of pictures, videos, images, text, and/or other information associated with the one or more interactive fields. Thumbnail(s) 301 may facilitate reviewing, recognizing, and/or organizing life moments, key events, locations, and/or people associated with a subject. By way of non-limiting example, selecting a first thumbnail from the thumbnail(s) 301 may direct a user to a corresponding event field 305.

Event field 305 may be configured to display key event information associated with one or more key events, including first key event information, second key event information, third key event information, and/or other key event information. The first key event information may include time and date information associated with a first key event, location information associated with the first key event, co-participants associated with the first key event, activities associated with the first key event, multimedia associated with the first key event, physiological information of the subject associated with the first key event, and/or other information associated with the first key event. The second key event information may include time and date information associated with a second key event, location information associated with the second key event, co-participants associated with the second key event, activities associated with the second key event, multimedia associated with the second key event, and/or other information associated with the second key event. The third key event information may include time and date information associated with a third key event, location information associated with the third key event, activities associated with the third key event, and multimedia associated with the third key event, and/or other information associated with the third key event.

Period field 310 may be configured to display one or more key events associated with a time range. In some embodiments, the time interval ranges may include a day, a week, a month, a year, and/or other intervals of time. Period field 310 may be configured to sort key event(s) by time, date, location, co-participants, heath data, emotional state, and/or other variables, in accordance with one or more embodiments. Period field 310 may be configured to display a timeline of key event(s). In some embodiments, period field 310 may be configured to display one or more thumbnail(s) 301 representing event fields 305 associated with one or more key events.

Location field 315 may be configured to display location information associated with key event(s) including geographical information and/or multimedia associated with the location of the key event(s). By way of non-limiting example, GPS data associated with a subject's location may be integrated with images and/or data from public databases (e.g., Google Street View images) to provide a more attractive user interface than the captured data alone. In another non-limiting example, key event(s) associated with a subject may be matched to news stories, personal social media feeds, public photos, and/or other data from external data source(s) relating to the location of key event(s) experienced by the subject. In some embodiments, location field 315 may be represented by one or more thumbnail(s) 301.

Friend field 320 may be configured to display friend information associated with one or more friends, family, caregivers, co-participants, and/or other people associated with the subject. Friend information may include multimedia associated with the friend, name of the friend, relationship to the friend, and/or other information about the friend. In some embodiments, the subject, caregiver, and/or other user(s) may input the friend information into the friend field 320. In some embodiments, friend field 320 may use facial recognition, voice recognition, and/or other data to identify friends, caregivers, and/or co-participants to populate the friend information automatically. In some embodiments, friend field 320 may be represented by one or more thumbnail(s) 301.

Health field 325 may be configured to display physiological data, biometric data, and/or other data associated with a subject for review by a caregiver. It is yet another aspect of one or more embodiments of the present disclosure to provide key event(s) information and/or other timeline data to one or more caregivers of the subject. Key event(s) presented by the GUI may provide convenient communication of information related to the health or wellbeing of the subject. By way of non-limiting example, a subject's personal data may be accessed by selecting icons (e.g., thumbnail(s) 301) on the GUI representing each event. Exemplary embodiments may alleviate problems related to sharing unprocessed data sets. By way of non-limiting example, the subject may send selected data directly to a caregiver for review (e.g., sharing data about outdoor activities with a fitness coach during an appointment). In some embodiments, life summaries may be useful for health coaching. A life summary may describe how active a subject is, how many people they interact with during the day, the types of locations the subject visits, how the subject travels to locations, and/or other life summary information. Life summaries may facilitate conversation between the subject and a caregiver in discussing lifestyle changes, factors contributing to unhealthy behavior, and/or other life factors relating to the subject. Health field 325 may be configured to facilitate sending health data to the caregiver responsive to a subject health measurement breaching a threshold level, responsive to a request from the caregiver, responsive to the subject selecting health data to send, and/or in response to other actions. Health field 325 may be configured to present the data succinctly for review by a caregiver via a list, a graph, a table, and/or other display. By way of non-limiting example, health field 320 may be configured to display the graph, the table, and/or other display responsive to a user selecting one or more thumbnail(s) 301 representing health field 325.

Routine field 330 may be configured to display daily, weekly, and/or monthly routine activities in which the subject participates. Routine field 330 may be configured to identify routine events from the life moment information and provide a reminder of the routine events to the subject. In some embodiments, routine field 330 may be configured for predictive analytics. Predictive analytics may be configured to suggest activities and/or provide a reminder to the subject based on detecting a subject's routine activities from past key event(s) and storing those routine activities to the timeline. By way of non-limiting example, responsive to the subject going to a dance class every Wednesday evening, the key event is detected as a routine event and a reminder notification may be sent to the subject prior to the next Wednesday evening dance class key event.

Emotion field 335 may be configured to display multimedia content based on a classification of the subject's mood (e.g., photographs recorded during positive, happy moments may be displayed such that bright colors are amplified and/or upbeat, happy music is playing, photographs recorded during negative, sad moments may be displayed in black and white). In some embodiments, the subject, caregiver, and/or other user(s) may input the physiological and/or emotional state of the subject into emotion field 335. By way of non-limiting example, a first step of emotion classification may include manually entering the physiological and/or emotional state of a subject associated with a life moment and/or key event. In another non-limiting example, emotion field 335 may prompt the subject and/or user to select between a set of emotions corresponding to identified key events. In some embodiments, emotion field 335 may be calibrated based on the emotions selected such that key events may be weighted for classification and/or categorization. By way of non-limiting example, responsive to a key event (e.g., the subject meeting with their caregiver) being selected as a happy event during calibration, similar events may be identified and weighted for happiness. Other weighted inputs may include voice, face recognition, physiological and/or psychological indicators such as galvanic skin response, heart rate, and/or film genre, and/or other weighted inputs. In some embodiments, one or more weighting inputs may be combined and/or calculated to produce an average of emotional status for a given type of key event. In some embodiments, emotion field 335 may be configured to assess the accuracy of the emotional classification of a key event. By way of non-limiting example, responsive to the subject being heard laughing while reviewing a key event, the weighting of the key event may be recalculated to indicate greater happiness.

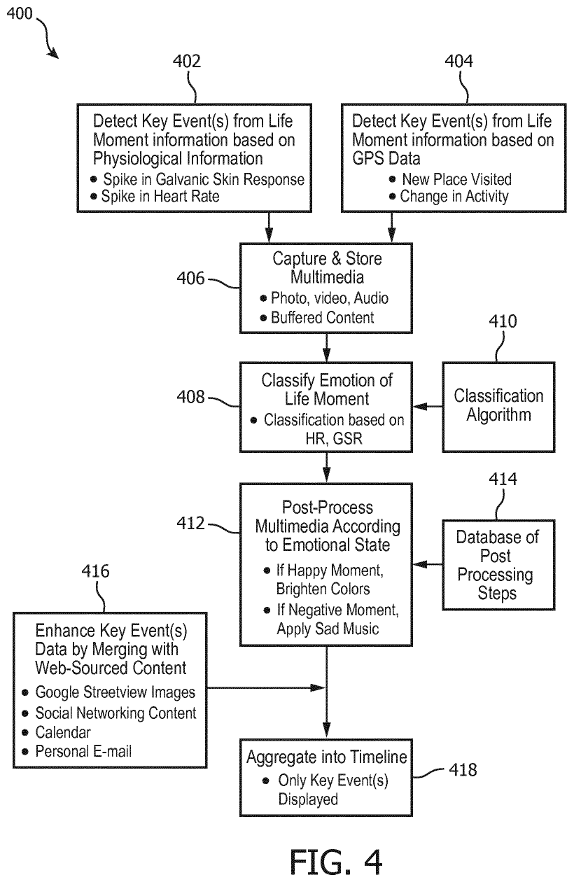

FIG. 4 illustrates an exemplary method 400 for capturing and presenting key events from life moment information, in accordance with one or more embodiments. The operations of exemplary method 400 presented below are intended to be illustrative. In some embodiments, exemplary method 400 may be accomplished with one or more additional operations not described, and/or without one or more of the operations discussed. Additionally, the order in which the operations of exemplary method 400 are illustrated in FIG. 4 and described below is not intended to be limiting.

In some embodiments, one or more operations of exemplary method 400 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of exemplary method 400 in response to instructions stored electronically on an electronic storage medium. The one or more processing devices may include one or more devices configured through hardware, firmware, and/or software to be specifically designed for execution of one or more of the operations of exemplary method 400.

At an operation 402, detect key event(s) based on subject physiological information breaching a threshold value. In some embodiments, detection of key event(s) may be responsive to subject biometric measurements such as increases in galvanic skin response (GSR), heart rate, and/or other physiological information. Operation 402 may be performed by one or more hardware processors configured to execute a machine-readable instruction component that is the same as or similar to life moment information component 118 (as described in connection with FIG. 1). Operation 402 may be facilitated by components that are the same as or similar to subject wearable sensors 206 and/or subject biometric sensors 208 (as described in connection with FIG. 2), in accordance with one or more embodiments.

At an operation 404, detect key event(s) based on a change in subject location information and/or subject activity. In some embodiments, key event(s) may be detected and/or identified based on GPS location data, accelerometer data identifying when subject movement or activity follows a stationary period, and/or other input signals. Operation 404 may be performed by one or more hardware processors configured to execute a machine-readable instruction component that is the same as or similar to life moment information component 118 (as described in connection with FIG. 1). Operation 404 may be facilitated by components that are the same as or similar to subject location sensors 212 (as described in connection with FIG. 2), in accordance with one or more embodiments.

At an operation 406, capture and store multimedia content in response to identification and/or detection of key event(s). In some embodiments, multimedia content may include images, photos, videos, sounds, audio recordings, text, animation, interactive content, and/or other multimedia. In some embodiments, buffered multimedia content (e.g., content recorded before, during, and/or after key event(s)) may be stored responsive to key event(s) being identified. Operation 406 may be performed by one or more hardware processors configured to execute a machine-readable instruction component that is the same as or similar to life moment information component 118 (as described in connection with FIG. 1). Operation 406 may be facilitated by components that are the same as or similar to subject multimedia sensors 210 (as described in connection with FIG. 2), in accordance with one or more embodiments.

At an operation 408, responsive to identifying key event(s) from the life moment information of a subject, classify the emotion of the subject (e.g., as negative, neutral, and/or positive). Classification may be based on biomedical information of the subject measured during the key event. In some embodiments, various types of wearable and/or passive sensors may be configured to provide output signals conveying the biomedical information to facilitate categorizing emotions such as angry, sad, happy, and/or other emotions. In some embodiments, biomedical information may be provided by biometric sensors including heart rate sensors, galvanic skin response sensors, skin conductance sensors, accelerometer, blood pressure sensors, EKG/cardiac monitors, pedometers, and/or other biometric sensors. In some embodiments, image and/or speech processing may be used to classify the emotion of persons encountered by the subject. Operation 408 may be performed by one or more hardware processors configured to execute a machine-readable instruction component that is the same as or similar to emotion classification component 123 (as described in connection with FIG. 1), in accordance with one or more embodiments.

At an operation 410, utilize a classification algorithm to classify subject and/or co-participant emotion. In some embodiments, the emotion classification algorithm may be an “out-of-the-box” algorithm, or an algorithm that requires calibration by a user. Operation 410 may be performed by one or more hardware processors configured to execute a machine-readable instruction component that is the same as or similar to emotion classification component 123 (as described in connection with FIG. 1). Operation 410 may be facilitated by components that are the same as or similar to external resources 124 (as described in connection with FIG. 1), in accordance with one or more embodiments.

At an operation 412, post-process multimedia content based on classification of the subject's emotional state. In some embodiments, post-processing of multimedia content may be based on a classification of the subject's mood (e.g., photographs recorded during positive, happy moments may be post-processed such that bright colors are amplified, videos recorded during negative, sad moments may be post-processed such that sad music is applied, and/or other multimedia post-processing according to emotional state). In some embodiments, post-processing may be based on the emotion of persons the subject interacted with. Operation 412 may be performed by one or more hardware processors configured to execute a machine-readable instruction component that is the same as or similar to emotion classification component 123 (as described in connection with FIG. 1), in accordance with one or more embodiments.

At an operation 414, access database for post-processing instructions. Operation 414 may be performed by one or more hardware processors configured to execute a machine-readable instruction component that is the same as or similar to life moment timeline component 120 (as described in connection with FIG. 1). Operation 414 may be facilitated by components that are the same as or similar to external resources 124 (as described in connection with FIG. 1), in accordance with one or more embodiments.

At an operation 416, enhance the presentation of key event(s) on the life moment timeline with web-sourced content. In some embodiments, the life moment information may be matched to web-sourced data to enhance the presentation to the user. By way of non-limiting example, GPS data associated with a subject's location may be integrated with images and/or data from public databases (e.g., Google Street View images) to provide a more attractive user interface than the captured data alone. In another non-limiting example, key event(s) associated with a subject may be matched to news stories, social networking content, calendar information, personal email, public photos, and/or other data from external data source(s) relating to the key event(s) experienced by the subject. The external data may be integrated in the display of the subject's key event(s) information for a more comprehensive reviewing experience. Operation 416 may be performed by one or more hardware processors configured to execute a machine-readable instruction component that is the same as or similar to life moment timeline component 120 (as described in connection with FIG. 1). Operation 416 may be facilitated by components that are the same as or similar to external data source(s) 108 (as described in connection with FIG. 1), in accordance with one or more embodiments.

At an operation 418, aggregate post-processed multimedia content into key event(s) information on the life moment timeline. In some embodiments, post-processing of multimedia content may include aggregating content from external data source(s) such as web search, mapping services, social networking sites, personal calendar and e-mail, and/or other data sources. By way of non-limiting example, the timeline may include a slideshow of pictures with background music, merged video footage, news and/or weather information, and/or other life moment information. In some embodiments, the life moment timeline is a life story book or diary of subject activities displayed on a graphical user interface. Operation 418 may be performed by one or more hardware processors configured to execute a machine-readable instruction component that is the same as or similar to life moment timeline component 120 (as described in connection with FIG. 1). Operation 418 may be facilitated by components that are the same as or similar to graphical user interface 110 (as described in connection with FIG. 1), in accordance with one or more embodiments.

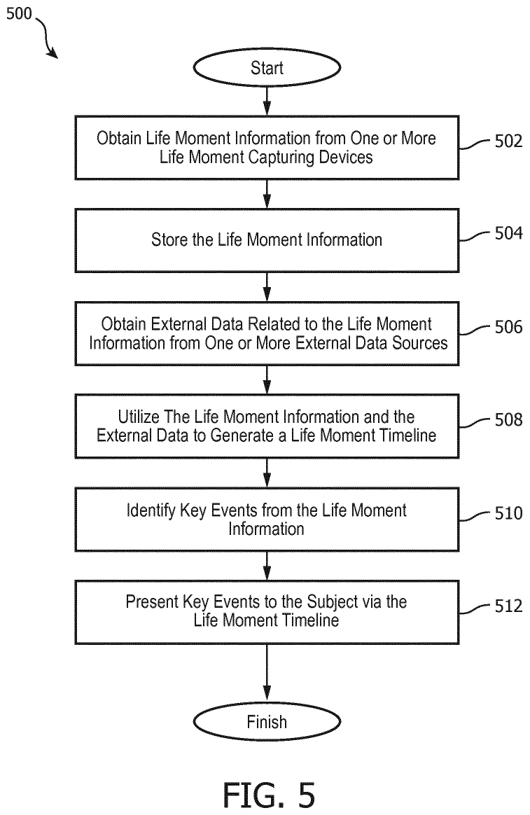

FIG. 5 illustrates a method 500 for capturing and presenting life moment information to a subject with cognitive impairment, in accordance with one or more embodiments. The operations of method 500 presented below are intended to be illustrative. In some embodiments, method 500 may be accomplished with one or more additional operations not described, and/or without one or more of the operations discussed. Additionally, the order in which the operations of method 500 are illustrated in FIG. 5 and described below is not intended to be limiting.

In some embodiments, one or more operations of method 500 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 500 in response to instructions stored electronically on an electronic storage medium. The one or more processing devices may include one or more devices configured through hardware, firmware, and/or software to be specifically designed for execution of one or more of the operations of method 500.

At an operation 502, obtain life moment information from one or more life moment capturing devices. In some embodiments, the life moment information includes information on daily activities experienced by the subject, including locations of the daily activities and/or co-participants in the daily activities. Operation 502 may be performed by one or more hardware processors configured to execute a machine-readable instruction component that is the same as or similar to life moment information component 118 (as described in connection with FIG. 1). Operation 502 may be facilitated by components that are the same as or similar to life moment capturing device(s) 106 (as described in connection with FIG. 1), in accordance with one or more embodiments.

At an operation 504, store the life moment information for later review by the subject and/or a caregiver of the subject. Operation 504 may be performed by one or more hardware processors configured to execute a machine-readable instruction component that is the same as or similar to life moment information component 118 (as described in connection with FIG. 1), in accordance with one or more embodiments.

At an operation 506, obtain external data relating to the life moment information from one or more external data sources. In some embodiments, the external data sources include one or more of public records, social media data sources, web-based data sources, current moment data sources, and/or other data sources. Operation 506 may be performed by one or more hardware processors configured to execute a machine-readable instruction component that is the same as or similar to life moment timeline component 120 (as described in connection with FIG. 1). Operation 506 may be facilitated by components that are the same as or similar to external data source(s) 108 (as described in connection with FIG. 1), in accordance with one or more embodiments.

At an operation 508, utilize the life moment information and the external data to generate a life moment timeline. In some embodiments, the life moment timeline is configured to display the life moment information to facilitate conversation between the subject and the caregiver. Operation 508 may be performed by one or more hardware processors configured to execute a machine-readable instruction component that is the same as or similar to life moment timeline component 120 (as described in connection with FIG. 1), in accordance with one or more embodiments.

At an operation 510, identify key events from the life moment information. Operation 510 may be performed by one or more hardware processors configured to execute a machine-readable instruction component that is the same as or similar to life moment information component 118 (as described in connection with FIG. 1), in accordance with one or more embodiments.

At an operation 512, present key events to the subject via the life moment timeline. In some embodiments, the life moment timeline is displayed on a graphical user interface. Operation 512 may be performed by one or more hardware processors configured to execute a machine-readable instruction component that is the same as or similar to life moment timeline component 120 (as described in connection with FIG. 1). Operation 512 may be facilitated by components that are the same as or similar to graphical user interface 110 (as described in connection with FIG. 1), in accordance with one or more embodiments.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the present disclosure has been described in detail for the purpose of illustration based on what is currently considered to be the most practical and preferred embodiments, it is to be understood that such detail is solely for that purpose and that the present disclosure is not limited to the disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

1. A conversation facilitation system configured to capture and present life moment information for a subject with cognitive impairment, the system comprising:
one or more hardware processors configured by machine-readable instructions to:
obtain life moment information from one or more life moment capturing devices, wherein the life moment information comprises information on daily activities experienced by the subject, including locations of the daily activities and/or co-participants in the daily activities;
store the life moment information to electronic storage for later review by the subject and/or a caregiver of the subject;
obtain external data relating to the life moment information from one or more external data sources, the external data sources comprising one or more of public records, social media data sources, web-based data sources, or current moment data sources; and
utilize the life moment information and the external data to generate a life moment timeline, wherein the life moment timeline is configured to display the life moment information, wherein the one or more hardware processors are configured such that the life moment timeline comprises one or more interactive fields, the interactive fields comprising one or more of an event field, a period field, a location field, a friend field, a health field, a routine field, or an emotion field.

2. (canceled)

3. The system of claim 1, wherein the one or more hardware processors are further configured by machine-readable instructions to:
identify key events from the life moment information; and
present key events to the subject via the life moment timeline, wherein the life moment timeline is displayed on a graphical user interface.

4. The system of claim 1, wherein the one or more hardware processors are further configured by machine-readable instructions to:
identify routine events from the life moment information; and
provide a reminder of the routine events to the subject via a graphical user interface.

5. The system of claim 1, wherein the one or more life moment capturing devices comprises a camera, a biometric sensor, and a location sensor.

6. The system of claim 1, wherein the one or more hardware processors are configured such that the life moment timeline displays key event information associated with key events identified from the life moment information, the key event information including:
first key event information, the first key event information comprising time and date information associated with a first key event, location information associated with the first key event, co-participants associated with the first key event, activities associated with the first key event, multimedia associated with the first key event, and physiological information of the subject associated with the first key event;
second key event information, the second key event information comprising time and date information associated with a second key event, location information associated with the second key event, co-participants associated with the second key event, activities associated with the second key event, multimedia associated with the second key event; and
third key event information, and the third key event information comprising time and date information associated with a third key event, location information associated with the third key event, activities associated with the third key event, and multimedia associated with the third key event.

7. The system of claim 1, wherein the one or more hardware processors are further configured by machine-readable instructions to process and/or post-process multimedia content based on classification of the subject's physiological and/or emotional state, wherein the subject's physiological and/or emotional state is identified based on biometric information of the subject, the biometric information being measured by the one or more life moment capturing devices.

8. A method for capturing and presenting life moment information to a subject with cognitive impairment, the method being performed by one or more hardware processors configured by machine-readable instructions, the method comprising:
obtaining life moment information from one or more life moment capturing devices, wherein the life moment information comprises information on daily activities experienced by the subject, including locations of the daily activities and/or co-participants in the daily activities;
storing the life moment information to electronic storage for later review by the subject and/or a caregiver of the subject;
obtaining external data relating to the life moment information from one or more external data sources, the external data sources comprising one or more of public records, social media data sources, web-based data sources, or current moment data sources; and
utilizing the life moment information and the external data to generate a life moment timeline, wherein the life moment timeline is configured to display the life moment information, wherein the life moment timeline comprises one or more interactive fields, the interactive fields comprising one or more of an event field, a period field, a location field, a friend field, a health field, a routine field, or an emotion field.

9. (canceled)

10. The method of claim 8, further comprising:
identifying key events from the life moment information; and
presenting key events to the subject via the life moment timeline, wherein the life moment timeline is displayed on a graphical user interface.

11. The method of claim 8, further comprising:
identifying routine events from the life moment information; and
providing a reminder of the routine events to the subject via a graphical user interface.

12. The method of claim 8, wherein the one or more life moment capturing devices comprises a camera, a biometric sensor, and a location sensor.

13. The method of claim 8, wherein the life moment timeline displays key event information associated with key events identified from the life moment information, the key event information including:
first key event information, the first key event information comprising time and date information associated with a first key event, location information associated with the first key event, co-participants associated with the first key event, activities associated with the first key event, multimedia associated with the first key event, and physiological information of the subject associated with the first key event;
second key event information, the second key event information comprising time and date information associated with a second key event, location information associated with the second key event, co-participants associated with the second key event, activities associated with the second key event, multimedia associated with the second key event; and
third key event information, and the third key event information comprising time and date information associated with a third key event, location information associated with the third key event, activities associated with the third key event, and multimedia associated with the third key event.

14. The method of claim 8, further comprising processing and/or post-processing multimedia content based on classification of the subject's physiological and/or emotional state, wherein the subject's physiological and/or emotional state is identified based on biometric information of the subject, the biometric information being measured by the one or more life moment capturing devices.

15.-21. (canceled)
